(19) United States
(12) Patent Application Publication
(10) Pub. No.: US 2004/0220131 A1
(43) Pub. Date: Nov. 4, 2004

(54) Method for treatment of cancerous angiogenic disorders

(75) Inventors: John K. Jackson, Vancouver (CA); Helen Burt, Vancouver (CA); Christopher Springate, Vancouver (CA); Martin Gleave, Vancouver (CA)

Correspondence Address: OPPEDAHL AND LARSON LLP, P O BOX 5068, DILLON, CO, 80435-5068, US

(73) Assignee: The University of British Columbia, Vancouver (CA)

(21) Appl. No.: 10/828,394

(22) Filed: Apr. 19, 2004

Related U.S. Application Data
(60) Provisional application No. 60/464,159, filed on Apr. 18, 2003.

(51) Int. Cl. A61K 48/00

(52) U.S. Cl. 514/44

(57) ABSTRACT

Cancerous angiogenesis-related diseases treated, and angiogenesis is reduced by administration of a therapeutically effective amount of a composition effective to reduce the effective amount of clusterin in an individual. Preferred therapeutic compositions comprise antisense oligonucleotides which reduce the effective amount of clusterin.

### BACKGROUND OF THE INVENTION

This application relates to a method for treatment of angiogenic disorders, and in particular cancerous angiogenic disorders.

Various cancers exhibit associated with angiogenesis which provides and enhanced blood flow to the cancer and facilitates its rapid growth. Among such cancers are colorectal, liver, renal, lung, breast, ovarian, prostate, brain, pancreas, stomach, and cervical cancers; some leukemias and lymphomas; and AIDS-related Kaposi's sarcoma. A therapeutic methodology which reduced or eliminated angiogenesis in individuals suffering from cancerous angiogenesis-related diseases would be desirable. It is an object of the present invention to provide such a methodology.

The present invention is based on the surprising finding that reduction in levels of clusterin leads to a reduction in angiogenesis. The glycoprotein clusterin was originally purified from ram rete testes fluid and sertoli cells and was reported to have cell aggregation properties (clustering) at these sites (Blaschuk et al. J. Biol. Chem. 258: 7714-20 (1983); Griswold, et al., Biochem. 25: 7265-70 (1986)). The protein was later found to be associated with Apolipoprotein A1 in plasma and was independently termed apolipoprotein J. Other names for the protein include sulphated glycoprotein-2 (SGP-2), complement cytolysis inhibitor (CCI) and testosterone repressed prostate messenger-2 (TRPM-2). The wide range of names reflects the diversity of tissue distribution and proposed functions for the protein. In fact, the protein has been shown to be present in most human tissues including prostate, testis, epidermis, kidney, uterus, liver spleen and brain and only absent in T lymphocytes (Grima, et al., Endocrinology 126: 2989-97 (1990)). Accordingly, clusterin has been proposed to be involved in many normal physiological functions in the body including lipid transportation (Burkey et al., J. Lipid Res. 33: 1517-26 (1992)), membrane turnover (Leger, et al., Biochem. Biophys. Res. Commun. 147: 196-203 (1987)), the inhibition of complement induced cytolysis and sperm maturation (Sylvester, et al., Biol. Of Reproduction 41: 941-8 (1989)). However, the specific mechanism(s) by which this protein functions in normal physiology remains to be elucidated.

An increased expression of clusterin has also been associated with many disease states, including cancer, athereosclerosis, myocardial infarction, kidney disease, and many neurological disorders (Sensibar, et al., Cancer Res. 55: 2431-7 (1995)). However, it is not known whether the increased expression of clusterin in such diseased tissues is part of the pathophysiology of the disease or merely a reaction to the disease process. Certainly there is a clear relationship between apoptotic cell death and clusterin expression whereby increased amounts of clusterin or clusterin expression (mRNA) are associated with a prosurvival signal in the relevant cells. Originally, it was reported that the increased expression of clustern was associated with cell survival within tissues regressing as a consequence of apoptosis. However, the primary role of clusterin in apoptotic control has been more recently described in many cells. For example, in prostate cancer cells, increased clusterin expression was shown to confer resistance to apoptotic cell death induced by either tumor necrosis factor (TNF-a) or hormone ablation (Sensibar, et al. 1995). In epidermal cancer cells, an increase in clusterin gene expression was shown to confer resistance to apoptotic cell death caused by heat shock and oxidative stress. Similarly, clusterin has been reported to protect granulosa cells from apoptotic cell death during follicular atresia.

The role of clusterin in the circulatory system has come under close scrutiny due to the presence of the protein in vascular endothelial cells, smooth muscle cells in arteries and atrial myocytes in the heart. It has been noted that the expression of clusterin is elevated in tissues undergoing remodeling following injury, such as myocardiocytes close to lesions in the heart. Although the exact role of clusterin in tissue repair is unknown, the protein may induce or promote phenotypic changes rather than general cell proliferation in cells involved in tissue remodeling.

In other cardiovascular diseases, increased clusterin expression in human vascular endothelial cells (HUVEC) is thought to confer resistance to the complement-induced activation of these cells which may be a proinflammatory signal in the pathogenesis of atherosclerosis. Also, the progression of premature vascular and thrombotic disease (atherothrombytic disease) disease is characterized by hyperhomocysteinemia. It is thought that one of the effects of elevated homocysteine levels may be to decrease the levels of the protective protein clusterin in vascular endothelial cells. In arterial graft failure due to anastomotic intimal hyperplasia, vascular endothelial cells are active participants because they migrate over the graft and the injured areas and secrete growth factors for vascular smooth muscle cells, thus contributing to the proinflammatory response at these disease sites. Clusterin expression was shown to be elevated at these disease sites and although clusterin was shown to inhibit the migration and adhesion of endothelial cells, it did not enhance or inhibit cell proliferation.

### SUMMARY OF THE INVENTION

The present invention provides a therapeutic in the form of a composition effective to reduce the effective amount of clusterin in an individual, and to a therapeutic method comprising the steps of administering to an individual suffering from the cancerous angiogenesis-related disease a therapeutically effective amount of a composition effective to reduce the effective amount of clusterin in the individual. Preferred therapeutic compositions comprise antisense oligonucleotides which reduce the effective amount of clusterin.

### BRIEF DESCRIPTION OF THE DRAWINGS

FIG. 1 shows cell viability of HUVECS following exposure to antisense in the presence and absence of paclitaxel.

FIG. 2 shows cell viability of HUVECS following exposure to antisense in the presence and absence of camptothecin.

FIG. 3 shows cell viability of HUVECS following exposure to antisense in the presence and absence of doxorubicin.

### DETAILED DESCRIPTION OF THE INVENTION

As used in the specification and claims of this application, the term “clusterin” refers to the glycoprotein originally derived from rat testes, and to homologous proteins derived from other mammalian species, including humans, whether denominated as clusterin or an alternative name. The sequences of numerous clusterin species are known. For example, the sequence of human clusterin is reported by Wong et al., Eur. J. Biochem. 221 (3), 917-925 (1994), and in NCBI sequence accession number NM_{—}001831 as the sequence of Seq. ID. No. 1 with the coding sequence spanning bases 48 to 1397.

As used in the specification and claims of this invention, an oligonucleotide consisting essentially of a specified sequence as reflected by a Seq. ID No. is an oligonucleotide with exactly the same sequence as that listed, or which differs from the exact sequence, for example as a result of the addition or substitution of one or two bases, but retains the ability to act as an antisense or RNAi agent to reduce the effective amount of clusterin. In the case of RNAi agents, the sequences given represents the sense si RNA strand, without the 3′-dTdT sequence, and the term consisting essentially of encompasses sequences including this deoxynucleotide tail.

The present invention provides a therapeutic composition, and methods for using such a composition for prevention of angiogenesis associated with cancerous angiogenesis-associated diseases. As used in this application, the term “cancerous angiogenesis-associated diseases” refers to cancerous diseases or conditions, wherein angiogenesis is observed as a symptom of the disease and facilitates cancer growth. Specific examples of such cancer include, without limitation, colorectal, liver, renal, lung, breast, ovarian, prostate, brain, pancreas, stomach, and cervical cancers; some leukemias and lymphomas; and AIDS-related Kaposi's sarcoma.

The therapeutic methods of the invention achieve a reduction in the effective amount of clusterin present in the individual being treated. As used in this application, the “effective amount of clusterin” is the amount of clusterin which is present in a form which is functional to enhance angiogenesis. The effective amount of clusterin may be reduced by decreasing the expression rate of clusterin, increasing the rate of clusterin degradation, or by modifying clusterin (for example by binding with an antibody) such that it is rendered inactive.

Reduction in the effective amount of clusterin may be accomplished by the administration of antisense oligodeoxynucleotides (ODNs), particularly antisense ODNs which are complementary to a region of the clusterin mRNA spanning either the translation initiation site or the termination site. The ODNs employed may be modified to increase the stability of the ODN in vivo. For example, the ODNs may be employed as phosphorothioate derivatives (replacement of a non-bridging phosphoryl oxygen atoms with a sulfur atom) which have increased resistance to nuclease digestion. MOE (2′-O-(2-methoxyethyl) modification (ISIS backbone) is also effective. Construction of such modified ODN is described in detail in US Patent Publication US 2003/0166591 A1 which is incorporated herein by reference. Specific antisense species which may be used in the method of the invention include, without limitation, those sequences listed as Seq. ID Nos. 2 to 15. Other antisense species which target expression of clusterin are described in U.S. Pat. No. 6,383,808, which is incorporated herein by reference.

Administration of antisense ODNs can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978 which are incorporated herein by reference. In general, the antisense is administered by intravenous, intraperitoneal, subcutaneous or oral routes, or direct local tumor injection.

Reduction of clusterin may also be accomplished using an RNAi approach. RNA interference or “RNAi” is a term initially coined by Fire and co-workers to describe the observation that double-stranded RNA (dsRNA) can block gene expression when it is introduced into worms (Fire et al. (1998) Nature 391, 806-811, incorporated herein by reference). dsRNA directs gene-specific, post-transcriptional silencing in many organisms, including vertebrates, and has provided a new tool for studying gene function. RNAi involves mRNA degradation, but many of the biochemical mechanisms underlying this interference are unknown. The use of RNAi has been further described in Carthew et al. (2001) Current Opinions in Cell Biology 13, 244-248, and Elbashir et al. (2001) Nature 411, 494-498, both of which are incorporated herein by reference. Clusterin expression can be reduced by the introduction of RNA molecules of about 21 to about 23 nucleotides that direct cleavage of clusterin-specific mRNA to which their sequence corresponds, referred to in the specification and claims of this application as RNAi agents. It is not necessary that there be perfect correspondence of the sequences, but the correspondence must be sufficient to enable the RNA to direct RNAi cleavage of the target mRNA. Specific useful RNA sequences for this purpose are set forth in Seq. ID Nos. 16-23, and sequences complementary thereto.

The RNAi agents of the invention are used in therapy to treat patients, including human patients, that have cancerous angiogenics diseases. siRNA molecules of the invention are administered to patients by one or more daily injections (intravenous, subcutaneous or intrathecal) or by continuous intravenous or intrathecal administration for one or more treatment cycles to reach plasma and tissue concentrations suitable for the regulation of the targeted mRNA and protein. The RNAi agent may be introduced as discrete siRNA molecules, or as part of an siRNA expression plasmid that results in the production of the RNAi agent in situ. In the latter case, sequences that contain the stated sequences and a complementary sequence separated by a loop region (for example of 9 bases) such that hairpin structures are formed and subsequently cleaved to form the RNAi agent may be employed.

In accordance with the invention, a therapeutic agent that reduces the effective amount of clusterin is administered to a subject, preferably a human subject, in need of treatment for a cancerous angiogenic disorder. The therapeutic agent is administered in an amount effective to result in a reduction of angiogenesis. It will be appreciated by persons skilled in the art that this amount will vary with the specific therapeutic agent, the route of administration and the type of carrier employed, if any. However, the determination of appropriate amounts is a matter of routine experimentation, and is generally defined by an upper limit determined based on toxicity, or a balancing of toxicity and efficacy.

Antisense oligonucleotides may be administered by normal means known to those skilled in the art such as by injection into the blood stream as a solution in an isotonic injection media. The injection regime may be by daily injection of a sufficient dose of the agent to maintain a therapeutic concentration of the oligonucleotide necessary for inhibition of cancer. Other parenteral routes include for example, intramuscular, intraperitoneal and subcutaneous. However, these agents may also be given orally using modern methods, known to those skilled in the art, to protect the oligonuclotides from degradation and enhance the passage of the oligonucleotides from the intestine to the blood stream.

These agents may also be delivered by other means more conducive to effective treatment of the cancer. For example it might be better to inject a solution of the antisense oligonucleotide directly into a disease site for example by intratumoral injection of a solution of the oligonucleotide. It is probable that larger (molecular weight) molecules such as proteins and oligonucleotides are cleared rather slowly from the tumour tissues joint so that an extended residence time in the tumor may allow greater penetration of the oligonucleotides into the target diseased cells. Also a much higher local concentration of the oligonucleotide may be achieved at the target site as compared to systemic routes of administration allowing for more effective treatment of the disease.

Controlled release drug delivery systems are particularly applicable to the effective treatment of cancer and the following examples illustrate the applicability of these systems. The oligonucleotides may be administered directly onto the tumor suspended in a biocompatible polymeric matrix such as a gel that released the agent in a controlled manner. The oligonucleotides might be encapsulated in microspheres made from, for example, poly lactic co glycolic acid and injected into target sites so that the oligonucleotides released from the microspheres by diffusion or as the biodegradable matrix broke down. Such microspheres might be injected directly into the tumor to allow for entrapment of the oligonucleotides in the tumor where they might release over a period of hours to days to months depending on the therapeutic need. Viscous gels such as those made from hyaluronic acid might be utilized for this purpose since this agent is mucoadhesive and may allow the oligonucleotide to be localized on the appropriate tissues, such as, for example around the site of surgical resection of a tumor to prevent regrowth of the tumor at that site. The positively charged biocompatible and biodegradable polysaccharide chitosan has been shown to be useful in binding and delivering oligonucleotides in vivo and this agent might be included in injectable formulations to allow for the controlled release at the site of the disease.

Chemoembolization is a process whereby the blood vessels leading to a tumor are blocked, preventing blood flow (and the supply of oxygen and nutrients to the tumor) and causing inhibiton of tumor growth augmented by use of chemotherapeutic drugs at the site of blockage. Microspheres containing oligonucleotides may be manufactured in the appropriate size range (e.g. 100 um in diameter) so that these may be injected into the blood stream and flow down into the tumor capillaries and become lodged in tumor tissues to stop blood supply and release the oligonucleotides at the site.

The therapeutic agent that reduces the effective amount of clusterin may be administered individually, or in combination with other compositions that inhibit angiogenesis (capillary growth), in either order or concurrently. Such compositions include, without limitation, antiproliferative drugs such as taxanes (e.g. paclitaxel), camptothecin and anti-angiogenic derivatives thereof, and doxorubicin which inhibit Huvecs in the low nanomolar range by the induction of apoptosis. As reflected in the results below, inhibition of cell proliferation induced by these antiproliferative drugs was enhanced by administration of antisense to produce downregulation of clusterin.

### EXAMPLE

HUVECS were grown for 2 days in wells after seeding at 1200 per well. Antisense oligonucleotide of Seq. ID No. 5 (4 μg/ml with lipofectin) was added in serum free medium and incubated with the cells for 4 hours. Then 100 μl of serum was added and incubation was continued overnight. The next day, 150 μl of drug solution in serum medium was added. After two days, 20 μl of mts solution was added and left for approximately 3 hours. Cell viability was determined as the difference between absorption at 490 and 595 nm.

Table 1 shows the measured absorbances for a first series of experiments in the which the drug tested was paclitaxel. The first row of results is the absorbance at 490 nm. The second row of results is the absorbance at 595 nm.

TABLE 1

| | | | | | | Drug | Drug | Drug | Drug |
|---|---|---|---|---|---|---|---|---|---|
| | AS | AS | AS | MM | | AS | AS | AS | MM |
| Control | 50 nM | 100 nM | 200 nM | 200 nM | Drug | 50 nM | 100 nM | 200 nM | 200 nM |
| 0.75 | 0.66 | 0.40 | 0.28 | 0.66 | 0.53 | 0.50 | 0.24 | 0.17 | 0.21 |
| 0.05 | 0.04 | 0.09 | 0.07 | 0.10 | 0.12 | 0.09 | 0.04 | 0.05 | 0.01 |

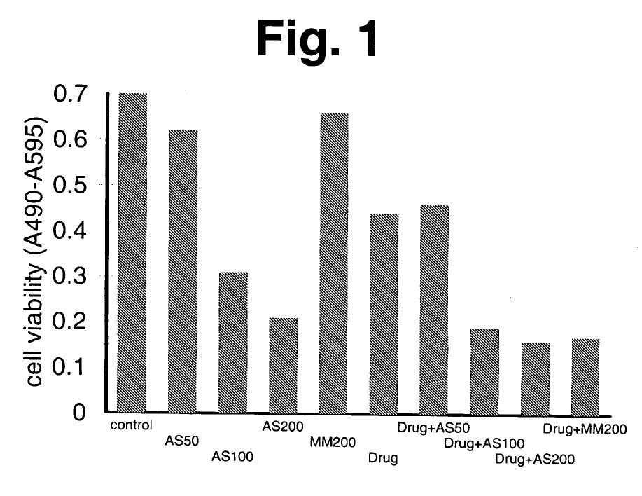

FIG. 1 shows the cell viability for each test sample in this set graphically. The bar in the center represents a mismatch (MM) control used at 200 nM. As shown, a dose dependent response to antisense concentration is observed, and the response is greater in the presence of 100 nM paclitaxel.

Table 2 shows the measured absorbances for a first series of experiments in the which the drug tested was camptothecin. The first row of results is the absorbance at 490 nm. The second row of results is the absorbance at 595 nm.

TABLE 2

| | | | | | | Drug | Drug | Drug | Drug |
|---|---|---|---|---|---|---|---|---|---|
| | AS | AS | AS | MM | | AS | AS | AS | MM |
| Control | 50 nM | 100 nM | 200 nM | 200 nM | Drug | 50 nM | 100 nM | 200 nM | 200 nM |
| 0.67 | 0.66 | 0.39 | 0.21 | 0.58 | 0.55 | 0.53 | 0.31 | 0.15 | 0.33 |
| 0.10 | 0.10 | 0.06 | 0.04 | 0.13 | 0.07 | 0.07 | 0.08 | 0.05 | 0.04 |

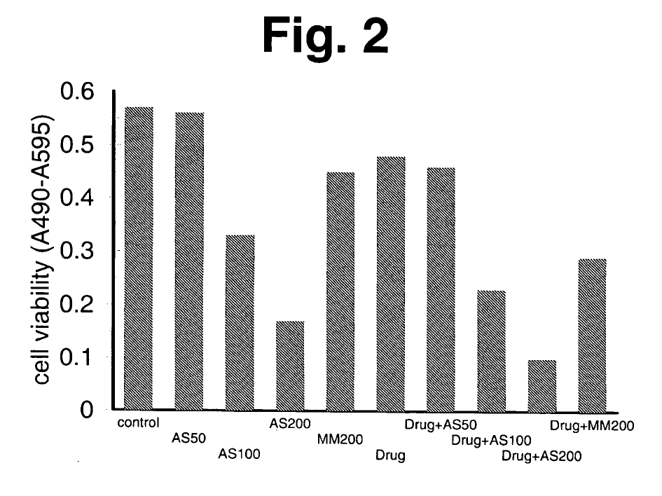

FIG. 2 shows the cell viability for each test sample in this set graphically. The bar in the center represents a mismatch (MM) control used at 200 nM. As shown, a dose dependent response to antisense concentration is observed, and the response is greater in the presence of 100 nM camptothecin.

Table 3 shows the measured absorbances for a first series of experiments in the which the drug tested was doxorubicin. The first row of results is the absorbance at 490 nm. The second row of results is the absorbance at 595 nm.

TABLE 3

| | | | | | | Drug | Drug | Drug | Drug |
|---|---|---|---|---|---|---|---|---|---|
| | AS | AS | AS | MM | | AS | AS | AS | MM |
| Control | 50 nM | 100 nM | 200 nM | 200 nM | Drug | 50 nM | 100 nM | 200 nM | 200 nM |
| 0.76 | 0.66 | 0.35 | 0.22 | 0.53 | 0.74 | 0.66 | 0.32 | 0.18 | 0.39 |
| 0.04 | 0.07 | 0.05 | 0.04 | 0.08 | 0.08 | 0.06 | 0.04 | 0.02 | 0.03 |

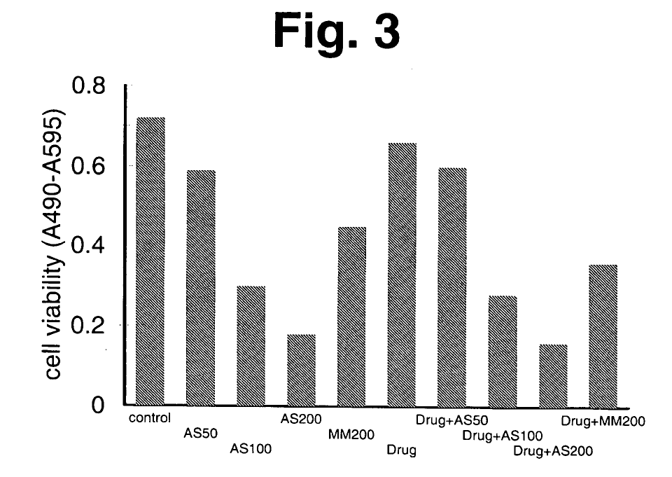

FIG. 3 shows the cell viability for each test sample in this set graphically. The bar in the center represents a mismatch (MM) control used at 200 nM. As shown, a dose dependent response to antisense concentration is observed, and the response is greater in the presence of 200 nM doxorubicin.

1. A method for treatment of a cancerous angiogenesis-related disease, comprising the steps of administering to an individual suffering from a cancerous angiogenesis-related disease an amount of a therapeutic composition effective to reduce the effective amount of clusterin in the individual.

2. The method of claim 1, wherein the therapeutic composition comprises an antisense oligonucleotide complementary to the sequence of human clusterin (Seq. ID. No. 1).

3. The method of claim 2, wherein the antisense oligonucleotide is selected from the group consisting of oligonucleotides whose sequence consists essentially of a sequence as set forth in Seq. ID Nos. 2-15.

4. The method of claim 1, wherein the therapeutic composition comprises an RNAi agent.

5. The method of claim 4, wherein the RNAi agent is selected from the group consisting of oligonucleotides whose sequence consists essentially of a sequence as set forth in Seq. ID Nos. 16 to 23 or a sequence complementary thereto.

6. A method for reducing angiogenesis in a cancerous angiogenesis-related disease, comprising the steps of treating cells of the cancer with amount of a therapeutic composition effective to reduce the effective amount of clusterin in the cells, and thereby to reduce the occurrence of angiogenesis.

7. The method of claim 6, wherein the therapeutic composition comprises an antisense oligonucleotide complementary to the sequence of human clusterin (Seq. ID. No. 1).

8. The method of claim 7, wherein the antisense oligonucleotide is selected from the group consisting of oligonucleotides whose sequence consists essentially of a sequence as set forth in Seq. ID Nos. 2-15.

9. The method of claim 6, wherein the therapeutic composition comprises an RNAi agent.

10. The method of claim 9, wherein the RNAi agent is selected from the group consisting of oligonucleotides whose sequence consists essentially of a sequence as set forth in Seq. ID Nos. 16 to 23 or a sequence complementary thereto.


SEQUENCE LISTING (23 sequences)

SEQ ID NO: 1 - DNA, 1676 bp, human
gaattccgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccacagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcaccttacgggggcatcttgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgaaaaaaaaaaaaaaaaaaaaaaaaaaggaattc

SEQ ID NO: 2 - DNA, 21 bp, human
gcacagcaggagaatcttcat

SEQ ID NO: 3 - DNA, 21 bp, human
gcacagcagcaggatcttcat

SEQ ID NO: 4 - DNA, 21 bp, human
tggagtctttgcacgcctcgg

SEQ ID NO: 5 - DNA, 21 bp, human
cagcagcagagtcttcatcat

SEQ ID NO: 6 - DNA, 21 bp, human
attgtctgagaccgtctggtc

SEQ ID NO: 7 - DNA, 21 bp, human
ccttcagctttgtctctgatt

SEQ ID NO: 8 - DNA, 21 bp, human
agcagggagtcgatgcggtca

SEQ ID NO: 9 - DNA, 21 bp, human
atcaagctgcggacgatgcgg

SEQ ID NO: 10 - DNA, 21 bp, human
gcaggcagcccgtggagttgt

SEQ ID NO: 11 - DNA, 21 bp, human
ttcagctgctccagcaaggag

SEQ ID NO: 12 - DNA, 21 bp, human
aatttagggttcttcctggag

SEQ ID NO: 13 - DNA, 21 bp, human
gctgggcggagttgggggcct

SEQ ID NO: 14 - DNA, 18 bp, human
tctcccggcttgcgccat

SEQ ID NO: 15 - DNA, 18 bp, human
tctcccggcatggtgcat

SEQ ID NO: 16 - DNA, 19 bp, human
ccagagcucgcccuucuac

SEQ ID NO: 17 - RNA, 19 nt, human
gaugcucaacaccuccucc

SEQ ID NO: 18 - RNA, 19 nt, human
cuaauucaauaaaacuguc

SEQ ID NO: 19 - RNA, 17 nt, artificial
  clusterin targeted siRNA
acaguuuuguugaauua

SEQ ID NO: 20 - RNA, 18 nt, human
augaugaagacuguccgc

SEQ ID NO: 21 - RNA, 20 nt, human
ugaaugaagggacuaaccug

SEQ ID NO: 22 - RNA, 20 nt, human
cagaaauagacaaagugggg

SEQ ID NO: 23 - RNA, 20 nt, human
acagagacuaagggaccaga